(19) United States
(12) Patent Application Publication
(10) Pub. No.: US 2023/0313098 A1
(43) Pub. Date: Oct. 5, 2023

(54) Columnar Cell Culture Vessel

(71) Applicants: SPL CO., LTD., Gyeonggi-do (KR); E-CELL CO., LTD., Gyeonggi-do (KR); INDUSTRY ACADEMY COOPERATION FOUNDATION OF SEJONG UNIVERSITY, Seoul (KR)

(72) Inventors: Sang Oh HEO, Gyeonggi-do (KR); Dong Hoon KIM, Gyeonggi-do (KR); Doo Hyun KIM, Seoul (KR); Duk Jae OH, Seoul (KR)

(73) Assignees: SPL CO., LTD., Gyeonggi-do (KR); E-CELL CO., LTD., Gyeonggi-do (KR); INDUSTRY ACADEMY COOPERATION FOUNDATION OF SEJONG UNIVERSITY, Seoul (KR)

(21) Appl. No.: 18/123,281

(22) Filed: Mar. 19, 2023

(30) Foreign Application Priority Data
Mar. 31, 2022 (KR) 10-2022-0040742

(51) Int. Cl. C12M 1/24; C12M 1/22; C12M 1/00; C12M 3/00

(52) U.S. Cl. CPC C12M 23/08; C12M 23/10; C12M 23/34; C12M 29/04; C12M 3/00; C12M 23/38

(57) ABSTRACT

A columnar cell culture vessel is disclosed. The columnar cell culture vessel according to an embodiment of the present disclosure may include: a cylindrical body having a chamber disposed therein, an inlet disposed through one side in an axial direction, and an outlet disposed through in the axial direction; a first cap coupled to the inlet and having an inflow port disposed therethrough; a second cap coupled to the outlet and having a discharge port disposed therethrough; and a support which is filled in the chamber and to which cells are attached to be cultured, wherein a culture solution is introduced into the chamber through the inflow port and discharged to the outside of the chamber through the discharge port.

### CROSS-REFERENCE TO RELATED APPLICATION

This application is based on and claims priority under 35 U.S.C. 119 to Korean Patent Application No. 10-2022-0040742, filed on Mar. 31, 2022, in the Korean Intellectual Property Office, the disclosure of which is herein incorporated by reference in its entirety.

### BACKGROUND OF THE INVENTION

### 1. Field of the Invention

The present disclosure relates to a columnar cell culture vessel and, more particularly, to a cell culture vessel used in a cell culture system in which a culture solution circulates.

### 2. Description of the Prior Art

The bioindustry is an industry that produces useful organisms or mass-produces substances that exist in extremely small amounts in nature by enhancing or improving organisms themselves or their unique functions, and in recent years, research thereinto is being actively conducted in pharmaceuticals, chemicals, foods, fibers, and the like.

Particularly, mass production of insulin, which is a special antidiabetic drug, and interferon, which is used for cancer treatment, has already been put into practical use in the field of pharmaceutical manufacturing by gene recombination technology, and biotech, that is, basic research on biotechnology is being conducted in the agricultural or chemical industries. Therefore, it is expected that increased food production and energy saving in chemical synthesis processes will be realized in the near future.

Technologies that form the basis of the bioindustry include genetic recombination technology in which only specific genetic information is extracted from a living organism and is inserted into the genes of fast-growing microorganisms (e.g., Escherichia coli), cell fusion technology, technology for culturing useful organisms in large quantities, and technology for bioreactor devices.

In the field of dual bioreactor devices, research and development are continuously being conducted on technologies that enable the cultivation of organisms to be performed under optimal conditions at the laboratory level while being monitored in real time. Accordingly, various types of biological culture devices have been released and used.

A vessel used for cell culturing includes a body and a lid so that a solution for cell culturing is accommodated in the vessel and is blocked from the outside.

In this regard, Korean Patent Publication No. 10-2021-0157046 discloses a cell incubator including a plate-shaped culture plate which has a culture surface on which cells are cultured and is surface modified such that the cells can be smoothly attached to the culture surface.

There is a difficulty in culturing cells in large quantity by using conventional cell culture vessels because of the limited culture area since cells are attached to the inner bottom surface to be cultured. In addition, the conventional cell culturing requires a separate work for exchanging a culture medium.

### SUMMARY OF THE INVENTION

One aspect of the present disclosure provides a columnar cell culture vessel in which a large quantity of cells can be cultured.

One aspect of the present disclosure provides a columnar cell culture vessel in which a culture solution can be circulated.

One aspect of the present disclosure provides a columnar cell culture vessel in which a cultured cell can be recovered.

In accordance with an aspect, a columnar cell culture vessel according to an embodiment of the present disclosure may include: a cylindrical body having a chamber disposed therein, an inlet disposed through one side in an axial direction, and an outlet disposed through the other side in the axial direction; a first cap coupled to the inlet and having an inflow port disposed therethrough; a second cap coupled to the outlet and having a discharge port disposed therethrough; and a support which is filled in the chamber and to which cells are attached to be cultured, wherein a culture solution is introduced into the chamber through the inflow port and discharged to the outside of the chamber through the discharge port.

In accordance with an aspect, the support of the columnar cell culture vessel according to an embodiment of the present disclosure may be a chip or bead made of a polymer or glass having a hydrophilic surface formed through plasma treatment.

In accordance with an aspect, the support of the columnar cell culture vessel according to an embodiment of the present disclosure may be a polymer chip or bead having a hydrophilic surface formed through protein or chemical coating.

In accordance with an aspect, the first cap or second cap of the columnar cell culture vessel according to an embodiment of the present disclosure may include: a cylindrical body having a thread disposed therein; a connection part coaxially protruding outward from the body and having a decreased diameter; a retention part coaxially protruding outward from the connection part and having a decreased diameter; and a connection port which coaxially protrudes outward from the retention part, has a decreased diameter, and has a Luer-lock tip protruding outward therefrom.

In accordance with an aspect, the first cap or second cap of the columnar cell culture vessel according to the embodiment of the present disclosure may further include a filter coupled inside the connection part to prevent the support from escaping from the chamber.

In accordance with an aspect, the first cap or second cap of the columnar cell culture vessel according to an embodiment of the present disclosure may further include a fixing ring coupled inside the body to prevent separation of the filter.

In accordance with an aspect, the first cap or second cap of the columnar cell culture vessel according to the embodiment of the present disclosure may have a protrusion protruding from the inside of the body to support the fixing ring.

In accordance with an aspect, the first cap or second cap of the columnar cell culture vessel according to the embodiment of the present disclosure may be replaced with a recovery cap, the recovery cap may be coupled to an entrance/exit port of a storage vessel for recovering the support, and the support may be moved to the storage vessel through the recovery cap.

In accordance with an aspect, the recovery cap of the columnar cell culture vessel according to an embodiment of the present disclosure may include: a cylindrical first body having a thread disposed therein; and a second body which coaxially protrudes outward from the first body, has an increased diameter, and has a thread disposed therein.

In accordance with an aspect, the second body of the columnar cell culture vessel according to the embodiment of the present disclosure may have a support ring coaxially protruding outward from the inside thereof and having a decreased diameter, and an entrance/exit of the storage vessel may be inserted between the first body and the support ring and be supported thereby.

In accordance with an aspect, the first cap or second cap of the columnar cell culture vessel according to the embodiment of the present disclosure may be replaced with a closure cap, the closure cap may seal the inlet or outlet, and the support may be carried or stored while being prevented from escaping from the chamber by the closure cap.

According to a columnar cell culture vessel according to an embodiment of the present disclosure, a cylindrical body having a chamber disposed therein, an inlet disposed through one side in an axial direction, and an outlet disposed through the other side in the axial direction; a first cap coupled to the inlet and having an inflow port disposed therethrough; a second cap coupled to the outlet and having a discharge port disposed therethrough; and a support which is filled in the chamber and to which cells are attached to be cultured, wherein a culture solution can be introduced into the chamber through the inflow port and discharged to the outside of the chamber through the discharge port, whereby the culture solution supplied from the outside can be continuously supplied to the chamber and no separate work for exchanging the culture medium is required.

According to the columnar cell culture vessel according to an embodiment of the present disclosure, the support may be a chip or bead made of a polymer or glass having a hydrophilic surface formed through plasma treatment, whereby a large quantity of cells can be cultured in a significantly enlarged surface for cell attachment.

According to the columnar cell culture vessel according to an embodiment of the present disclosure, the support may be a chip or bead made of a polymer or glass having a hydrophilic surface formed through protein or chemical coating, whereby a large quantity of cells can be cultured in an enlarged surface for cell attachment.

According to the columnar cell culture vessel according to an embodiment of the present disclosure, the first cap or second cap may include: a cylindrical body having a screw thread therein; a connection part coaxially protruding outward from the body and having a decreased diameter; a retention part coaxially protruding outward from the connection part and having a decreased diameter; and a connection port which coaxially protrudes outward from the retention part, has a decreased diameter, and has a Luer-lock tip protruding outward therefrom, whereby the inflow tube and the discharge tube of the culture solution can be safely connected to each other and the culture solution can be stably circulated.

According to the columnar cell culture vessel according to an embodiment of the present disclosure, the first cap or second cap may further include a filter coupled inside the connection part to prevent the support from escaping from chamber, whereby the support can be prevented from moving by the flow of a culture solution.

According to the columnar cell culture vessel according to an embodiment of the present disclosure, the first cap or second cap may further include a fixing ring coupled inside the body to prevent separation of the filter, whereby a state in which the filter is coupled inside the body can be stably maintained.

According to the columnar cell culture vessel according to an embodiment of the present disclosure, the first cap or second cap may have a protrusion protruding from the inside of the body to support the fixing ring, whereby a state in which the fixing ring is coupled inside the body can be stably maintained.

According to the columnar cell culture vessel according to an embodiment of the present disclosure, the first cap or second cap may be replaced with a recovery cap, the recovery cap may be coupled to an entrance/exit port of a storage vessel for recovering the support, and the support may be moved to the storage vessel through the recovery cap, whereby the support in which cells have been cultured can be stably recovered.

According to the columnar cell culture vessel according to an embodiment of the present disclosure, the recovery cap may include: a cylindrical first body having a thread disposed therein; and a second body which coaxially protrudes outward from the first body, has an increased diameter, and has thread disposed therein, whereby the recovery cap can be stably coupled to the storage vessel.

According to the columnar cell culture vessel according to an embodiment of the present disclosure, the second body may have a support ring disposed therein and coaxially protruding outward while having a decreased diameter, and an entrance/exit of the storage vessel may be inserted between the first body and the support ring and be supported thereby, whereby, the recovery cap can be more stably coupled to the storage vessel.

According to the columnar cell culture vessel according to an embodiment of the present disclosure, the first cap or second cap may be replaced with a closure cap, the closure cap may seal the inlet or outlet, and the support may be carried or stored while being prevented from escaping from the chamber by the closure cap, whereby the support can be carried or stored while being safely accommodated in the chamber.

### BRIEF DESCRIPTION OF THE DRAWINGS

The above and other aspects, features, and advantages of the present disclosure will be more apparent from the following detailed description taken in conjunction with the accompanying drawings, in which:

FIG. 1 is a perspective view of a columnar cell culture vessel according to an embodiment of the present disclosure;

FIG. 2 is an exploded perspective view of a columnar cell culture vessel according to an embodiment of the present disclosure;

FIG. 3 is an exploded cross-sectional view of a columnar cell culture vessel according to an embodiment of the present disclosure;

FIG. 4 is an exploded perspective view of a columnar cell culture vessel including a recovery cap according to an embodiment of the present disclosure;

FIG. 5 is a cross-sectional view showing a recovery cap being used according to an embodiment of the present invention;

FIG. 6 is a perspective view of a columnar cell culture vessel including a closure cap according to an embodiment of the present disclosure;

FIG. 7 is a perspective view of a cell culture system in which a columnar cell culture vessel is used according to an embodiment of the present disclosure; and

FIG. 8 is a circuit diagram of a cell culture system in which a columnar cell culture vessel is used according to an embodiment of the present disclosure.

### DETAILED DESCRIPTION OF THE EXEMPLARY EMBODIMENTS

Hereinafter, preferred embodiments of the present disclosure will be described in detail with reference to the accompanying drawings.

However, in describing the present disclosure, descriptions of already known functions or configurations will be omitted to clarify the gist of the present disclosure.

Up, down, left and right, front, back, and the like which refer to directions in the description and claims of the invention, etc., are not intended to limit the use of rights and are determined based on the relative position between the drawings and configurations for convenience of description. Each direction described below is based on this, except for cases specifically described otherwise.

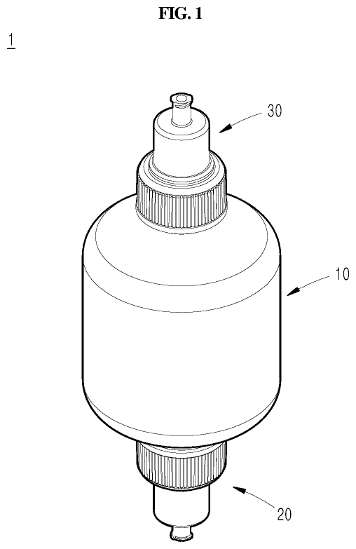

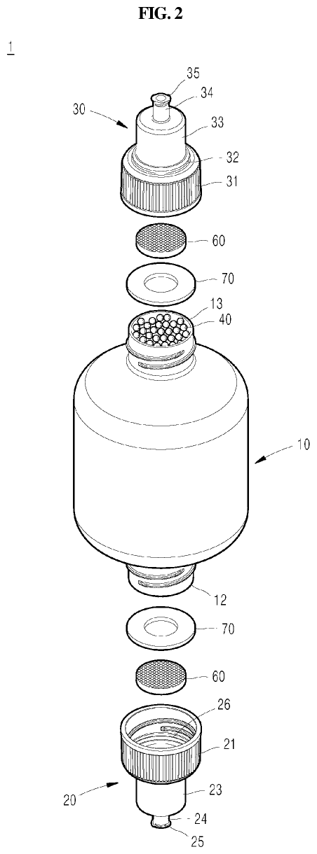

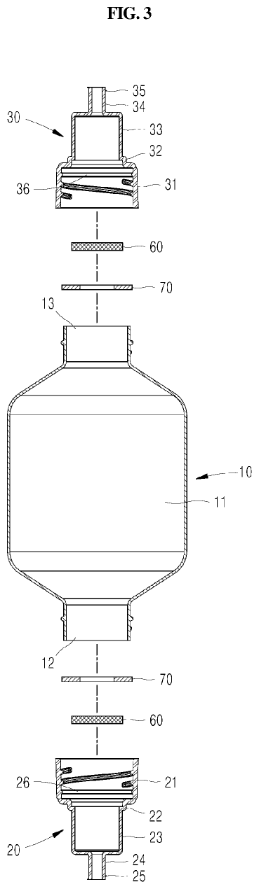

FIGS. 1 to 3, the columnar cell culture vessel 1 according to a preferred embodiment of the present disclosure may include: a cylindrical body 10 having a chamber 11 disposed therein, an inlet 12 disposed through one side in an axial direction, and an outlet 13 disposed through the other side in the axial direction; a first cap 20 coupled to the inlet 12 and having an inflow port disposed therethrough; a second cap 30 coupled to the outlet 13 and having a discharge port disposed therethrough; and a support 40 which is filled in the chamber 11 and to which cells are attached to be cultured.

A culture solution may be introduced into the chamber 11 through the inflow port and discharged to the outside of the chamber 11 through the discharge port.

The culture solution refers to a liquid culture medium.

The culture medium may be mainly composed of nutrients required by cells to culture the cells, and substances for special purposes may be added to be mixed therewith if necessary.

The culture medium may supply water, which is indispensable for survival and growth, as well as carbon sources, nitrogen sources, inorganic salts, growth factors (vitamins), and the like as nutrients, and various nutrients, osmotic pressure, pH, etc. may be adjusted according to the type of cells to be cultured.

The columnar cell culture vessel 1 may enable culture solution supplied from the outside to be continuously supplied into and discharged from the chamber 11, eliminate the need for a separate work for exchanging a culture solution after use, and secure an enlarged cell attachment surface. Therefore, the columnar cell culture vessel 1 can provide an optimal environment for culturing a large quantity of cells.

Referring to FIGS. 1 to 3, the body 10 is a component configured to provide the chamber 11 in which cells are cultured.

The body 10 has a cylindrical structure in which both sides disposed in the axial (longitudinal) direction are open.

The body 10 may have the inlet 12 disposed through one side (lower side) in the axial direction and the outlet 13 disposed through the other side (upper side) in the axial direction.

The inlet 12 and the outlet 13 of the body 10 may be used interchangeably.

The body 10 may be formed vertically symmetrically.

The body 10 may have a diameter decreasing toward the bottom thereof and protrude, and then further protrude to form the inlet 12 to which the first cap 20 is coupled.

The body 10 may have a diameter decreasing toward the top thereof and protrude, and then further protrude to form the outlet 13 to which the second cap 30 is coupled.

The inlet 12 and the outlet 13 may have threads disposed on the outer circumferential surface thereof.

The body 10 may be manufactured using a substance including a polystyrene (PS) resin with excellent moldability, shape stability, electrical insulation, and chemical resistance, a PVC (polyvinyl chloride) resin, a PC (polycarbonate) resin or a PP (polypropylene) resin with excellent chemical resistance, electrical insulation, insulation, and corrosion resistance through injection molding.

The body 10 may be formed of a transparent substance so that the state of the chamber 11 can be visually checked from the outside.

Referring to FIGS. 1 to 3, the first cap 20 is a component configured to connect an inflow tube to the inlet 12 of the body 10.

The inflow tube may be a passage through which the culture solution supplied from an external culture medium part moves.

The inflow tube may be coupled to the inflow port of the first cap 20.

The first cap 20 may include: a cylindrical body 21 having threads disposed therein; a connection part coaxially protruding outward from the body 21 and having a decreased diameter; a retention part 23 coaxially protruding outward from the connection part and having a decreased diameter; and a connection port 24 which coaxially protrudes outward from the retention part 23, has a decreased diameter, and has a Luer-lock tip 25 protruding outward therefrom.

The diameter of the first cap 20 may be reduced toward the body 21, the connection part, the retention part 23, and the connection port 24.

The first cap 20 may be formed in a stepped shape in which the body 21, the connection part, the retention part 23, and the connection port 24 coaxially protrude outward.

The first cap 20 may further include a filter 60 coupled inside the connection part to prevent the support 40 from escaping from the chamber 11.

The first cap 20 may further include a fixing ring 70 coupled inside the body 21 to prevent escaping of the filter 60.

The body 21 may be a part screw-coupled to the inlet 12 and may have a screw thread disposed on the inner circumferential surface thereof and a plurality of irregularities disposed along on the outer circumferential surface thereof to be spaced apart from each other.

The body 21 may have a connection part protruding from a part for blocking the inlet 12.

The body 21 may have an annular protrusion 26 protruding from the inside thereof to support a fixing ring 70.

The protrusion 26 may maintain a state in which the fixing ring 70 presses and supports a filter 60.

The connection part is a part for connecting the body 21 and the retention part 23.

The connection part may have the disk-shaped filter 60 disposed therein to be coupled thereto.

The filter 60 may prevent the support 40 accommodated in the chamber 11 from escaping from the chamber 11.

The fixing ring 70 supported by the protrusion 26 of the body 21 may prevent the filter 60 from escaping from the connection part.

The retention part 23 is a part for providing a space where a culture solution from the inflow port stays before moving to the chamber 11.

The connection port 24 may be a part corresponding to the inflow port of the first cap 20, and an inflow tube may be coupled thereto.

The connection port 24 may have the Luer-lock tip 25 protruding to prevent separation of the inflow tube.

The Luer-lock tip 25 may be formed as a screw thread protruding from the outer circumferential surface of the connection port 24.

The inflow tube may be connected to the connection port 24 by an adapter (not shown) coupled to the Luer-lock tip 25.

The adapter may have a screw thread coupled to the Luer-lock tip 25.

The first cap 20 may include a control valve (not shown).

The control valve may be a manual valve and may prevent a culture solution or the support 40 from escaping through the connection port when the columnar cell culture vessel 1 is replaced.

Referring to FIGS. 1 to 3, the second cap 30 is a component configured to connect the outlet 13 of the body 10 and the discharge tube.

The discharge tube may be a passage through which a culture solution to be supplied to the chamber 11 moves to an external culture medium part.

The discharge tube may be coupled to the discharge port of the second cap 30.

The second cap 30 may include: a cylindrical body 31 having threads disposed therein; a connection part 32 coaxially protruding outward from the body 31 and having a decreased diameter; a retention part 33 coaxially protruding outward from the connection part 32 and having a decreased diameter; and a connection port 34 which coaxially protrudes outward from the retention part 33, has a decreased diameter, and has a Luer-lock tip 35 protruding outward therefrom.

The diameter of the second cap 30 may be reduced toward the body 31, the connection part 32, the retention part 33, and the connection port 34.

The second cap 30 may be formed in a stepped shape in which the body 31, the connection part 32, the retention part 33, and the connection port 34 coaxially protrude outward.

The second cap 30 may further include a filter 60 coupled inside the connection part 32 to prevent the support 40 from escaping from the chamber 11.

The second cap 30 may further include a fixing ring 70 coupled inside the body 31 to prevent escaping of the filter 60.

The body 31 may be a part screw-coupled to the outlet 13 and may have a screw thread disposed on the inner circumferential surface thereof and a plurality of irregularities disposed along on the outer circumferential surface thereof to be spaced apart from each other.

The body 31 may have a connection part 32 protruding from a part for blocking the outlet 13.

The body 31 may have an annular protrusion 36 protruding from the inside thereof to support a fixing ring 70.

The protrusion 36 may maintain a state in which the fixing ring 70 presses and supports a filter 60.

The connection part 32 is a part for connecting the body 31 and the retention part 33.

The connection part 32 may have the disk-shaped filter 60 disposed therein to be coupled thereto.

The filter 60 may prevent the support 40 accommodated in the chamber 11 from escaping from the chamber 11.

The fixing ring 70 supported by the protrusion 36 of the body 31 may prevent the filter 60 from escaping from the connection part 32.

The retention part 33 is a part for providing a space where a culture solution to be supplied to the chamber 11 stays before moving to the discharge port.

The connection port 34 may be a part corresponding to the discharge port of the second cap 30, and the discharge tube may be coupled thereto.

The connection port 34 may have the Luer-lock tip 35 protruding to prevent separation of the discharge tube.

The Luer-lock tip 35 may be formed as a screw thread protruding from the outer circumferential surface of the connection port 34.

The discharge tube may be connected to the discharge port by an adapter (not shown) coupled to the Luer-lock tip 35.

The adapter may have a screw thread coupled to Luer-lock tip 35.

Referring to FIGS. 1 to 3, the support 40 is a component configured to provide an environment in which cells may be attached to the support 40 to be cultured.

The support 40 may be a chip or bead made of polymer or glass and filled in the chamber 11 and may provide an environment in which a large quantity of cells are attached to the chip or bead to be cultured.

The support 40 may be a chip or bead made of polystyrene, agarose, methylcellulose, hyaluronan, or a combination thereof.

The support 40 may be a chip or bead made of glass.

The support 40 may have a spherical, elliptical, or irregular shape.

The support 40 may be treated to provide a hydrophilic surface to facilitate attachment to adherent cells.

The support 40 may be surface modified through vacuum or atmospheric pressure plasma treatment to have a hydrophilic surface.

The support 40 may be formed to have a hydrophilic surface through a protein or chemical coating.

The support 40 may be coated with collagen, fibrin, fibronectin, vitronectin, Matrigel, gelatin, laminin, heparin, or poly-lysine, an extracellular matrix, or a combination thereof

The support 40 may contain a physiologically active substance or may have a surface coated with a biologically active substance.

The biologically active substance may be selected from the group consisting of basic fibroblast growth factor (bFGF), brain-derived neurotrophic factor (BDNF), vascular endothelial cell growth factor (VEGF), and interleukin (IL)-4, IL-6, IL-2, EGF, epidermal growth factor (EGF), stem cell factor (SCF), interferon gamma (IFNγ), and granulocyte macrophage colony-stimulating factor (GM-CSF).

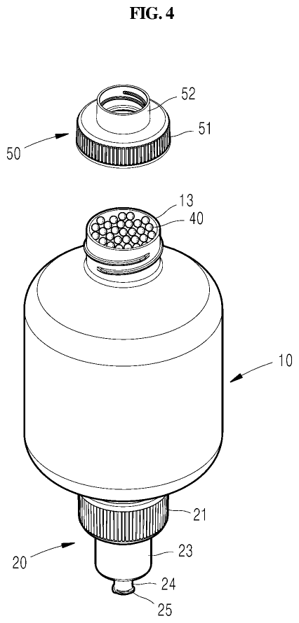

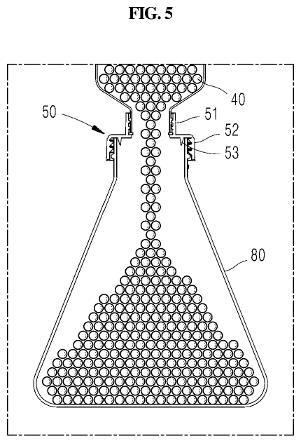

Referring to FIGS. 4 and 5, when cell culturing is finished, the first cap 20 or second cap 30 may be replaced with a recovery cap 50.

The recovery cap 50 is a component for recovering the support 40 filled in the chamber 11 to a storage vessel 80.

The recovery cap 50 may have one side disposed in the axial direction which is coupled to the inlet 12 or outlet 13 of the body 10, and the other side disposed in the axial direction which is coupled to an entrance/exit port of the storage vessel 80.

The recovery cap 50 may have a passage through which the support 40 may move in the axial direction, and thus the support 40 may move to the storage vessel 80 through the passage of the recovery cap 50.

The recovery cap 50 may include: a cylindrical first body 51 having a thread disposed therein; and a second body 52 which coaxially protrudes outward from the first body 51, has an increased diameter, and has a screw thread disposed therein.

The first body 51 is a part coupled to the inlet 12 or the outlet 13 of the body 10, and the second body 52 is a part coupled to the entrance/exit port of the storage vessel 80.

The diameter of the entrance/exit port of the storage vessel 80 may be formed to be larger than the diameter of the inlet 12 or the outlet 13 of the body 10 so that the support 40 can be smoothly recovered thereto, and accordingly, the second body 52 may be formed to have a larger diameter than the first body 51.

The second body 52 may have a support ring 53 coaxially protruding outward from the inside thereof and having a decreased diameter.

The entrance/exit port of the storage vessel 80 may be inserted between the first body 51 and the support ring 53 and supported thereby.

The first body 51 may be screw-coupled to the outer circumferential surface of the entrance/exit port of the storage vessel 80, and the support ring 53 may be in close contact with the inner circumferential surface of the entrance/exit port of the storage vessel 80.

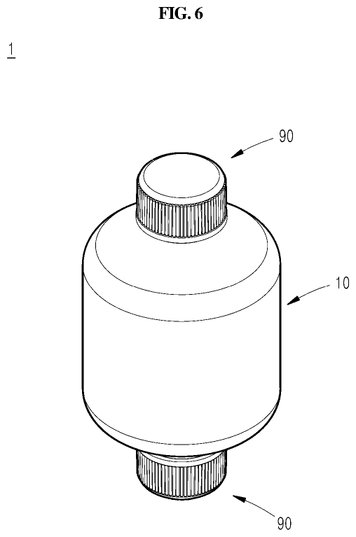

Referring to FIG. 6, when cell culturing is finished, the first cap 20 or second cap 30 may be replaced with a closure cap 90.

The closure cap 90 is a component configured to carry or store the support 40 filled in the chamber 11.

The closure cap 90 may be coupled to the inlet 12 of the body 10 to seal the inlet 12.

The closure cap 90 may be coupled to the outlet 13 of the body 10 to seal the outlet 13.

When the closing cap 90 is coupled, the support 40 may be carried or stored while being prevented from escaping from the chamber 11 by the closing cap 90.

If necessary, one of the first cap 20 or second cap 30 may be replaced with the closure cap 90 and the other may be replaced with the recovery cap 50 and then recovering the support 40 to the storage vessel 80 may be performed.

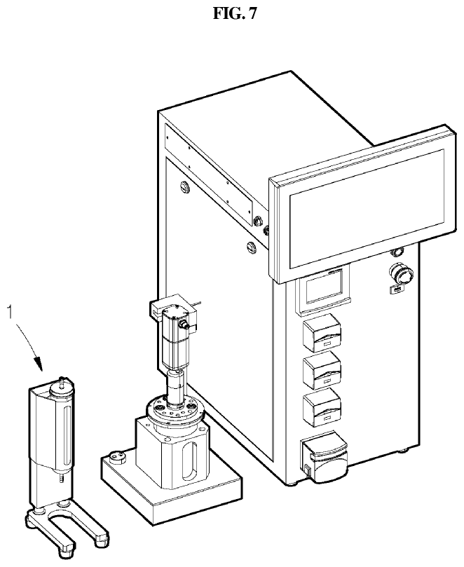

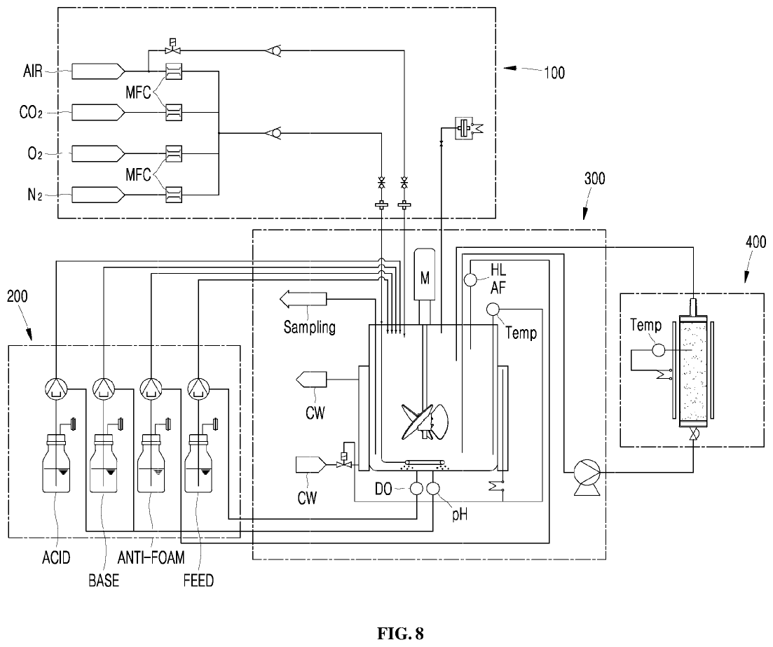

Referring to FIGS. 7 and 8, the circulation type cell culture system in which the columnar cell culture vessel 1 is used may include: a gas part 100 configured to supply nitrogen, oxygen, carbon dioxide, and air; a liquid part 200 configured to supply an acidic liquid, a basic liquid, an antifoaming liquid, and a feed liquid; a culture medium part 300 configured to generate and supply a culture solution in which the components supplied from the gas part 100 and the liquid part 200 are mixed; a culture part 400 configured to culture cells by using a culture solution; and a controller 500 configured to control composition, production, and supply of the culture solution.

The circulation type cell culture system is composed of the gas part 100, the liquid part 200, the culture medium part 300, the culture part 400, and a controller 500, and the controller 500 may be configured to circulate a culture solution between the culture medium part 300 and the culture part 400 to adjust the culture of cells.

Referring to FIGS. 7 and 8, the gas part 100 is a component configured to supply gas components constituting a culture solution to the culture medium part 300.

The gas part 100 may supply nitrogen, oxygen, carbon dioxide, and air to the culture medium part 300.

The gas part 100 may include a gas mass flow controller (MFC), and the controller 500 may operate the gas mass flow controller to adjust the supply of nitrogen, oxygen, carbon dioxide, and air to be supplied to the culture medium part 300.

Referring to FIGS. 7 to 8, the liquid part 200 is a component configured to supply liquid components constituting a culture solution to the culture medium part 300.

The liquid part 200 may supply acidic liquid, basic liquid, defoaming liquid, and feed liquid to the culture medium part 300.

The liquid part 200 may include a pump, and the controller 500 may operate the pump to adjust the supply of acidic liquid, basic liquid, defoaming liquid, and feed liquid to be supplied to the culture medium part 300.

Referring to FIGS. 7 and 8, the culture medium part 300 is a component configured to mix the gas component supplied from the gas part 100 and the liquid component supplied from the liquid part 200 to generate a culture solution and supply the culture solution to the culture part 400.

The culture medium part 300 may include: a culture medium vessel; a stirrer; and a sensor part having at least one of a pH measuring sensor for measuring the hydrogen ion concentration of the culture solution, a first temperature sensor for measuring the temperature of the culture solution, and an anti-foam sensor for measuring bubbles in the culture solution.

The controller may adjust the composition, production, and supply of a culture solution by using information transmitted from the DO sensor, pH sensor, first temperature sensor, and anti-foam sensor, so that the cell culture environment can be monitored in real time and thus be maintained in an optimal state.

Referring to FIGS. 7 and 8, the culture part 400 is a component configured to culture cells by using a culture solution supplied from the culture medium part 300.

The culture part 400 may include a columnar cell culture vessel 1 filled with a support 40 to which cells are attached to be cultured.

The controller 500 may supply a culture solution generated in the culture medium vessel to the columnar cell culture vessel 1 through the inflow tube and recover the culture solution used in the columnar cell culture vessel 1 to the culture medium vessel through the discharge tube. Therefore, the culture solution can be continuously supplied to the columnar cell culture vessel 1.

The columnar cell culture vessel 1 may be connected to the culture medium vessel of the culture medium part 300 through the inflow tube and the discharge tube.

The circulation type cell culture system may enable a culture solution to be continuously supplied to the columnar cell culture vessel 1 of the culture part 400, eliminate the need for a separate work for exchanging a culture solution, enable a large quantity of cells to be efficiently cultured, and automate a cell culturing process.

The culture part 400 may further include a second temperature sensor configured to measure the temperature of a culture solution; and a second heater configured to heat the columnar cell culture vessel 1.

The controller 500 may adjust the temperature of the columnar cell culture vessel 1 by using information transmitted from the second temperature sensor, and therefore, the cell culture environment can be monitored in real time and thus be maintained in an optimal state.

The controller 500 may operate the second heater, based on the information transmitted from the second temperature sensor to heat the columnar cell culture vessel 1, and therefore, the columnar cell culture vessel 1 can be constantly maintained at an optimal temperature.

The culture part 400 may further include a recovery device configured to ultrasonically vibrate the columnar cell culture vessel 1.

The recovery device may separate the cells completely cultured by ultrasonic vibration from the support 40.

The controller 500 may operate the recovery device to separate the completely cultured cells from the support 40, and therefore, a cell separation operation can be efficiently performed.

The control unit 500 may provide an optimal culture environment according to the type of cells to be cultured while monitoring the cell culture environment in real time and adjust the composition, production, and supply of the culture solution to automate mass cell culturing.

In the foregoing, although specific embodiments of the present disclosure have been described and shown, the present disclosure is not limited to the described embodiments, and it is common knowledge to those skilled in the art that various modifications and variations can be made without departing from the spirit and scope of the present disclosure.

Therefore, such modifications or variations should not be individually understood from the technical spirit or viewpoint of the present disclosure, and modified embodiments should be said to belong to the claims of the present disclosure.
(Government-sponsored R&D Project)
(Project ID) 1425143347
(Project No.) S2840580
(Ministry Involved) Ministry of SMEs and Startups
(Responsible Institute) Korea Technology & Information Promotion Agency for SMEs
(R&D Business Name) Innovative R&D of SMEs
(R&D Project Name) Development of column-type automated cell culture system for mass production of adhesive therapeutic cells
(Contribution Rate) 1/1
(Institute Conducting Project) SPL LIFE SCIENCES
(R&D Period) 2020.07.16 ˜ 2024.07.15

[Descriptions of Symbols]

| | |
|---|---|
| 1: Columnar cell culture vessel | 10: Body |
| 11: chamber | 12: Inlet |
| 13: Outlet | 20: First cap |
| 21: Body | 22: Connection part |
| 23: Retention part | 24: Connection port |
| 25: Luer-lock tip | 26: Protrusion |
| 30: Second cap | 31: Body |
| 32: Connection part | 33: Retention part |
| 34: Connection port | 35: Luer-lock tip |
| 36: Protrusion | 40: Support |
| 50: Recovery cap | 51: First body |
| 52: Second body | 53: Support ring |
| 60: Filter | 70: Fixed ring |
| 80: Storage vessel | 90: Closure cap |
| 100: Gas part | 200: Liquid part |
| 300: Culture medium part | 400: Culture part |
| 500: Controller | |

What is claimed is:

1. A columnar cell culture vessel comprising:
a cylindrical body having a chamber disposed therein, an inlet disposed through one side in an axial direction, and an outlet disposed through other side in the axial direction;
a support which is filled in the chamber and to which cells are attached to be cultured;
a first cap coupled to the inlet and having an inflow port disposed therethrough; and
a second cap coupled to the outlet and having a discharge port disposed therethrough,
wherein a culture solution is introduced into the chamber through the inflow port and discharged out of the chamber through the discharge port.

2. The columnar cell culture vessel of claim 1, wherein the support is a chip or bead made of a polymer or glass having a hydrophilic surface formed through plasma treatment.

3. The columnar cell culture vessel of claim 1, wherein the support is a polymer chip or bead having a hydrophilic surface formed through protein or chemical coating.

4. The columnar cell culture vessel of claim 1, wherein the first cap or second cap comprises:
a cylindrical body having a thread disposed therein;
a connection part coaxially protruding outward from the body and having a decreased diameter;
a retention part coaxially protruding outward from the connection part and having a decreased diameter; and
a connection port coaxially which protrudes outward from the retention part, has a decreased diameter, and has a Luer-lock tip protruding outward therefrom.

5. The columnar cell culture vessel of claim 4, wherein the first cap or second cap further comprises:
a filter coupled inside the connection part to prevent the support from escaping from the chamber: and
a fixing ring coupled inside the body to prevent separation of the filter.

6. The columnar cell culture vessel of claim 5, wherein the first cap or second cap comprises a protrusion protruding from the inside of the body to support the fixing ring.

7. The columnar cell culture vessel of claim 1, wherein the first cap or second cap is replaceable with a recovery cap,
the recovery cap is couplable to an entrance/exit port of a storage vessel for recovering the support, and
the support is movable to the storage vessel through the recovery cap.

8. The columnar cell culture vessel of claim 7, wherein the recovery cap comprises:
a cylindrical first body having a thread disposed therein; and
a second body which coaxially protrudes outward from the first body, has an increased diameter, and has a thread disposed therein.

9. The columnar cell culture vessel of claim 8, wherein the second body has a support ring coaxially protruding outward from the inside thereof and having a decreased diameter, and
the entrance/exit of the storage vessel is inserted between the first body and the support ring and is supported thereby.

10. The columnar cell culture vessel of claim 1, wherein the first cap or second cap is replaceable with a closure cap,
the closure cap is configured to seal the inlet or outlet, and
the support is carriable or storable while being prevented from escaping from the chamber by the closure cap.
